(19) United States
(12) Patent Application Publication
(10) Pub. No.: US 2024/0366103 A1
(43) Pub. Date: Nov. 7, 2024

(54) ENHANCED RF MEDICAL IMAGING AND IDENTIFICATION SYSTEM

(71) Applicant: Pavlo Anatoliyovich Molchanov, Las Cruces, NM (US)

(72) Inventor: Pavlo Anatoliyovich Molchanov, Las Cruces, NM (US)

(21) Appl. No.: 18/144,027

(22) Filed: May 5, 2023

(51) Int. Cl. A61B 5/05; A61B 5/00; G01S 13/44; G01S 13/89

(52) U.S. Cl. CPC A61B 5/05; A61B 5/7257; G01S 13/449; G01S 13/89

(57) ABSTRACT

Method and apparatus for enhance medical imaging and identification with potential image resolution up to 1000 times smaller than diffraction limit, wavelength. Recording of wavefront of good body penetrating Radio Frequency (RF) electromagnetic waves in form of digital hologram allows to recreate 3 dimensional images of objects. Reference signals in set of monopulse antennas with overlapping squinted beams provides enhanced image resolution and allows suppression of scattering medium influence. Transforming of reflected near field signals to frequency-space-time domains allows identification of objects and scattering medium by spectrum signatures. Direct digitizing and digital interface for connection to multi-channel signal processor allows loose distributing of transceiver antenna modules around object or in small space.

### REFERENCES CITED

### US Patent Documents:

U.S. Pat. No. 5,384,573 January 2095 Terry M. Turpin

U.S. Pat. No. 5,734,347 March 2098 E. Lee McEligot

US 2019/0310347 A1 October 2019 Stephen A. Harman

US 2017/0082732 A2 March 2017 Gordon K. Oswald

U.S. Pat. No. 0,035,783 A1 February 2014 Pavlo A. Molchanov 342/357.59

U.S. Pat. No. 3,906,505 September 1975 Stephen E. Lipsky 343/119

### OTHER PUBLICATIONS

1. Stephen A. Harman “A comparison of staring radars with scanning radars for UAV detection: introduction of the Alarm™ staring radar”, 2015, European Radar Conference (EURAD), EUMA, September 2015, 185-188, XP032824534.
2. Stephen A. Harman, (Aveillant Ltd., Cambridge, U.K.), “Holographic Radar Development”, 2021 Feb. 7 (microwavejournal.com).
3. S. E. Lipsky, “Microwave Passive Direction Finder”, SciTech Publishing Inc. Raleigh, NC 27613, 2004.
4. P. Molchanov, A. Gorwara, “Fly Eye Radar Concept”. IRS2017. International Radar Symposium, Prague, July 2017.

### INTRODUCTION

Diffraction limits maximal image resolution depending on wavelength. High frequency short wavelength millimeter-wave radars provide relatively good imaging resolution, but poor body penetration. The low penetration requires increasing radar transmitting power which leads to health dangers. Long wavelengths, such as meter or centimeter-wave radars, provide good body penetration with small transmitting power, from a few to tens milliwatts (like cell phones) but provide low image resolution (from centimeters to meters). As result the application of RF imaging radars for small imaging resolution is directly limited by the diffraction limit.

Military radars can provide directional accuracy smaller than 0.01 degrees for wavelength 6 cm. This corresponds to a resolution of 20 micrometers at a distance of 1 meter, which is enough to illuminate through the human body. A monopulse radar system with overlapping squinted beams allows for the 10-100 fold enhancement of directional accuracy. This enables the possibility of medical imaging resolution smaller than 2 microns for low power RF signals (not more than ten milliwatts) which do not represent any health risk.

### OBJECT OF THE INVENTION

Object of invention is RF enhanced medical imaging system which provides high resolution imaging, 2-4 orders better than diffraction limit with low power RF signals. Second object is the identification of internal body tissues and objects and scattering medium components by spectrum signatures.

### FIELD OF THE INVENTION

This invention is not primarily an image processing technology. It is a medical imaging technology based on the application of radio frequency signals for the imaging and dentification of objects and tissue components. It is about the application of long wavelength radio frequency radar technology for creating high resolution images. Proposed imaging radar based on processing of reflected from object near field wavefront signals recorded as digital hologram with wide, as minimum one object covering antenna array with reference to processor time. Monopulse processing of antenna signals with overlapping antenna patterns will enhance directional and image resolution. The multi-axis distribution of overlapping antennas and the application of reference antennas will provide maximum information about object shape from a few sides for object and medium component identification. Direct on antenna digitizing and digital interface for connection to a multi-channel signal processor allows for the loose distribution of transceiver antenna modules around the object or in a small space.

### SUMMARY OF THE INVENTION

Proposed RF enhanced imaging and identification radar is an integration of a holographic simultaneous wide covering non-scanning system with monopulse high directional accuracy method of multi-beam multi-axis fast multi-channel signals processing. Radar system with multi-beam multi-axis overlapping antenna patterns with staring, not scanning continuous wide covering area allows for the reception of maximum 3 dimensional (3D) information from the whole object or multiple objects and scattering medium. Multi-axis 3D object observation provides high-resolution and high probability of object recognition. High-speed object imaging provided by simultaneous multi-channel signals processing with one-step algorithms and reference signals from overlapping antennas. Digitizing of reflected signals directly on each directional antenna allows distribution of transceiver antenna modules (TAM) around observation area or in small volume space. Transformation and processing of received signals in time domain, frequency domain and multi-axis space domain provides the additional possibility to enhance image resolution and object identification.

### BACKGROUND OF THE INVENTION

RF enhanced resolution medical imaging radar system is based on receiving near-field wavefront of reflected signals and processing not only amplitudes, but phase and frequency shifted near-field components. If the object is illuminated with coherent continuous wave (CW) signals, reflected signals in far field will be coherent and have same frequency and wavelength corresponding to Cittert-Zernike theorem. Wavefronts do not allow reconstruction of object shape in the far field (FIG. 1). Wavefronts in the near field consist of information about reflected waves as time and phase shifts and allow for the reconstruction of object shape (number of ducks in picture) and size of object. Moreover, the dielectric coefficient of the object creates additional proportional phase shift (like in Impedance Smith Chart), which provides information allowing for the identification of the object. Transform of reflected time-space domain signals to frequency domain allows to filter frequency components comprising information about shape, size, and dielectric coefficient and create spectrum signature of the object. The dispersion of continuous waves creates additional frequency components (FIG. 2) which correspond to object size and shape in near field. These frequency components are not harmonics. Frequency of components lay in 5-20% range from transmitting frequency. Antenna array is arranged as an array of directional antennas covering the area of observation or area of possible objects with overlap of as minimum one axis antenna patterns creating antenna sub-arrays in each axis. FIG. 3 shows antenna patterns overlapping in axis X, but patterns can be overlapping in axis Y. U. W. Multi-axis overlapping antennas provide better image resolution and additional information about the object. The monopulse method of direction finding with overlapping antennas provides better accuracy of direction finding and imaging resolution. Overlapping antenna patterns presented in FIG. 4 in polar coordinates shows that monopulse processing provides better accuracy of phase measurement and does not need base distance between the antennas. Regular phase measurement with half wavelength base distance between antennas will increase antenna array size for low RF range.

Diagram of system for recording digital hologram is presented in FIG. 5. A continuous wave radio frequency electromagnetic transmission covers all of area of observation or possible location of the object. Reflected, and/or partially penetrated, from all object electromagnetic signals receiving by one or array of antennas positioned in near field area. Amplitude, time delay and phase delay of frequency shifted signal components consists of 3 dimensional information about the entire object and are combined in antenna (or antenna array) and can be directly digitized and recorded as a digital hologram, relative to processor time. Processor time serves as the reference signal for the hologram. The near field reflected wavefront consists of information about shape, size and impedance of the object and medium.

Signal processor transforming digital holographic data to frequency domain (FIG. 6), where frequency components consisting of information about shape, size, dielectric properties of object and medium can be grouped, filtered and coded. Transferred to the frequency domain these frequency components can be visualized on a display as a “Waterfall”, real time dynamic image.

Frequency components need be transferred back to time domain, for example by inverse Fourier transform and the object can then be reconstructed in time-frequency-space domains (FIG. 7) in two or multi-axis coordinates. One recording directional antenna array or multiple antenna array must simultaneously cover all the object or area of observation. Scanning antenna systems with a narrow beam, not covering all the object at once record only direct reflected signals and are losing signals reflected outside of the antenna angle of view. The main condition for recording real digital holograms is real optical hologram recording of the wavefront of signals reflected from all objects (object beam) relative to reference beam. Some existing imaging radar systems are using scanning of narrow beam antennas around object for recording a hologram. This can be considered quasi-holograms but real digital holograms cannot be recorded by a scanning antenna because it is not covering all of the object simultaneously. The loss of signals outside of the narrow antenna pattern is not compensated by scanning the antenna around the object surface. As result the recovered image loses part of the information about the object and also loses resolution. A wider area of observation can be covered and a digital hologram can be recorded by multiple antenna arrays positioned in two (FIG. 8) or more axes. Antennas elements positioned in two and three axes are shown in FIG. 9.

Note, that recording of a digital hologram provides for an increase image resolution which is no longer limited by diffraction limit but rather defined by the digitizing (sampling) frequency and transmitting and sampling frequencies and phase stability.

Image synthesis using time sequential holography method and apparatus proposed in U.S. Pat. No. 5,384,573, wherein reflections that travel in phase to the observation location: producing two coherent radiation beams, directing the two beams onto a receiving plane provided with an array of radiation receiving cells and storing output signals from each receiving cell, and controlling the two beams. Instead of two beams radar analog of the optical hologram (U.S. Pat. No. 5,734,347) recording a radar image in the range/doppler plane, the range/azimuth plane, and/or the range/elevation plane. The invention embodies a means of modifying the range doppler data matrix by scaling, weighing, filtering, rotating, tilting, or otherwise modifying the matrix to produce some desired result. But scanning beams with the sequential recording of signals does not receive signals reflected from all parts of the object(s) as in real optical holograms. Losing these parts of the signal degrades the resolution of the object's image.

S. Harman in his paper “A comparison of staring radars with scanning radars for UAV detection” [2] proposed staring radars having multiple static receiver beams that do not scan and are constantly sensing. The fundamental difference between staring and scanning radars is that for a staring radar the transmit beam is stationary and fully filled with potentially multiple static receiver beams. The number of receiver beams within the transmit floodlight determines the gain on reception and the ability to localize in angle. The key opportunity that staring radars offer is the ability to give exceptional information and utility e.g., optimal detection, tracking and target ID capability in an optimal time. This is due to the ability to set the processing dwell time for a given task or alternatively work using multiple parallel processes each having different processing dwell times. Clearly this technique is unachievable in mechanically scanned surveillance radars or conventional phased array radars that can only afford to schedule short periods of time to a given sector in order to maintain complete coverage with acceptable latency.

To reach maximal accuracy of direction finding Lipsky S.E. [3] proposed an antenna array with a plurality of fixed, narrow beamwidth antennas, geographically oriented to provide omnidirectional coverage, as set of antennas is selected. It presents an explanation of the monopulse method for microwave direction finding with two pairs of directional antennas, positioned by azimuth and elevation boresight. The general theory of the monopulse method considers that the angle sensing function falls into one of three categories: amplitude, phase, or a combination developed by combining their sum and difference (2-4). The phase angle difference, as measured in each antenna, compared against the arriving signal phase front, is denoted as ψ. The difference in signal path length is defined by the equation, S=D sin φ, which depends on the antenna aperture displacement (spatial angular shift) D. Letting φ be the phase lag caused by the difference in the time of arrival between two signals gives:

$$ψ = - 2 π \frac{S}{λ} = - 2 π \frac{D sin φ}{λ} , \qquad ( 1 )$$

where:
φ—the angle of arrival measured from bore sight; λ—the wavelength.

If A and B are RF voltages measured at the reference boresight and incident antennas, respectively, then

$$A = M sin ( ω t ) \qquad ( 2 )$$

$$and$$

$$B = M sin ( ω t + ψ ) = M sin ( ω t - \frac{2 π D}{λ} sin φ ) , \qquad ( 3 )$$

where M is a common constant defined by signal power. This shows that the angle of arrival φ is contained in the RF argument or phase difference of the two beams for all signals off the boresight axis. Direction finding by way of amplitude comparison methods can provide a root mean square (RMS) accuracy smaller than 2° in 100 ns after a direct wave arrives. High accuracy phase measurements provide high accuracy and fast direction finding. Most importantly, the monopulse method does not require time consuming (from millisecond for small operations to tens of seconds for FFT (Fast Fourier Transform)), computer calculations to provide critical information about targets position, speed, and identity.

The combination of staring antenna array with high directional accuracy monopulse method and digital processing and synchronization is proposed in a paper by P. Molchanov, A. Gorwara, “Fly Eye Radar Concept” [4].

### PRIOR ART

In the patent application “System for Optical Imaging with Unshifted Reference Beam”, US 2021/0153742 A1, May 27, 2021, Edgar Emilio Morales Delgado, (PRIOR ART FIG. 1) proposed a system or a device for imaging includes an infrared light source, an optical structure, and a sensor. The sensor may include an image pixel array. The infrared light generates an infrared illumination signal for illuminating a diffuse medium such as tissue. An optical structure is configured to facilitate an interference of an infrared reference beam and an infrared exit signal. The infrared reference beam has a same infrared wavelength as the infrared illumination signal. The image pixel array is configured to capture holographic infrared images of the interference of the infrared reference beam and the infrared exit signal. The penetration of infrared signals into human tissues is insufficient for many imaging applications and IR optical systems are not inexpensive for now.

Qianqian Fang, in his U.S. Pat. No. 7,825,667 B2 from Nov. 2, 2010, “Microwave imaging system and processes, and associated software products” (PRIOR ART FIG. 2) proposed a microwave imaging process, and a system controlled by an associated software product, illuminate a target with microwaves from a transmitting antenna. Receiving antennas receive microwaves scattered by the target, and form microwave data. The illumination and receiving repeat over multiple transmitting antennas and multiple microwave frequencies. The microwave data is processed to form permittivity and conductivity images by selecting a background dispersion model for permittivity and conductivity. Permittivity and conductivity dispersion coefficients are determined, and permittivity and conductivity distributions are calculated, for each of the microwave frequencies. Forward solutions at multiple frequencies are determined from property distributions, and a dispersion coefficient based Jacobian matrix is determined. Dispersion coefficient updates are determined using the microwave data, and the dispersion coefficients are updated. Permittivity and conductivity distributions are recalculated for each of the frequencies, and the forward solutions are determined at multiple frequencies from property distributions. This system provides good accuracy of object detection with switching antenna beams that illuminate the entire search space but construction of a multi-input multi-output (MIMO) antenna array not possible for some applications.

In U.S. Pat. No. 8,494,615 B2, “Apparatus and method for doppler-assisted MIMO radar microwave imaging” proposed by Raviv Melamed, PRIOR ART FIG. 3. Invention provides a method and apparatus for enhanced microwave imaging of an object collects microwave responses for multiple combinations of transmit antennas, receive antennas, and object movement states. The responses are grouped into sets of responses corresponding to at least two object movement states. An image is reconstructed from the set of responses for each movement state, and a differential image representative of object movement is generated from the reconstructed image for each of the at least two object movement states. The differential image is overlaid on a reconstructed image to obtain an enhanced composite image of the object. The proposed invention can provide images with resolution limited by diffraction limit, approx. wavelength. Such wavelength resolution is not enough for some applications of RF systems.

### BRIEF DESCRIPTION OF DRAWINGS

PRIOR ART FIG. 1 shows a known system for optical imaging with an unshifted reference beam.

PRIOR ART FIG. 2 shows a known microwave imaging system and process.

PRIOR ART FIG. 3 shows a known doppler-assisted MIMO radar for microwave imaging.

FIG. 1 shows a picture and diagram for Near Field diffraction waves components explanations.

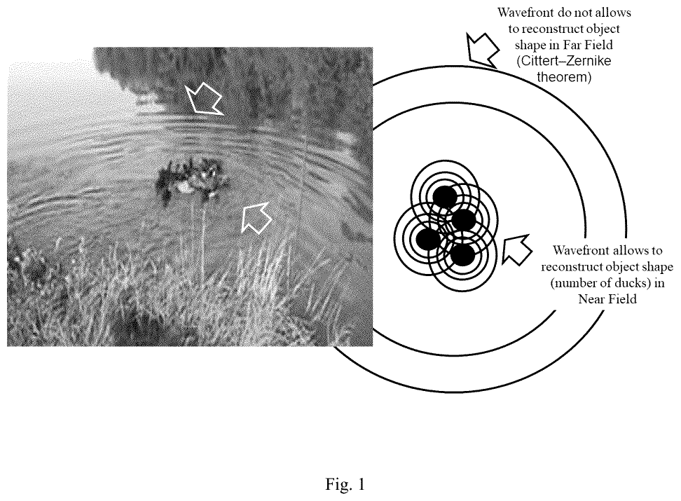

FIG. 2 shows a diagram demonstrating diffraction of water waves in the Near Field.

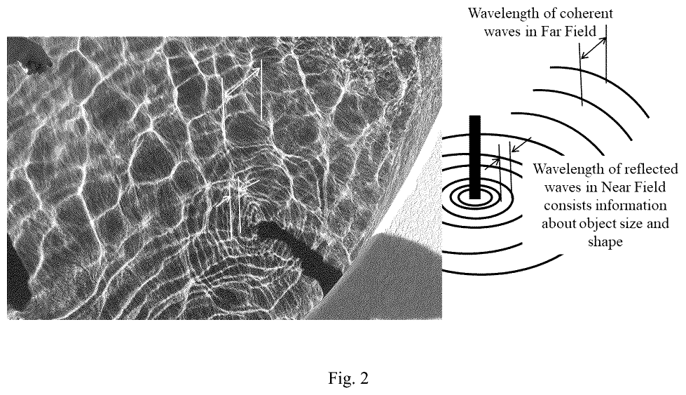

FIG. 3 shows a diagram of an antenna array with overlapping antenna patterns in one axis.

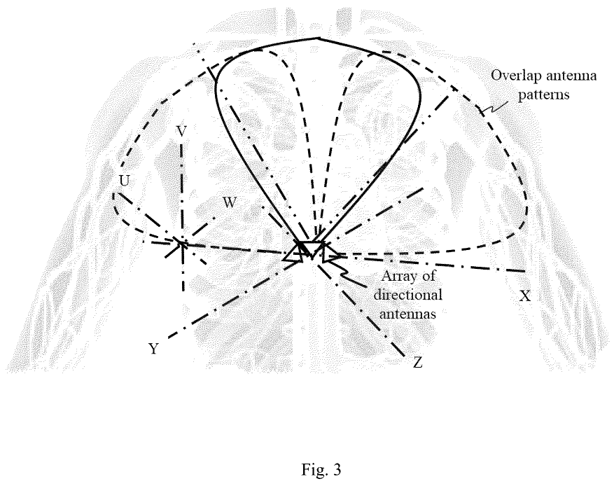

FIG. 4 shows a phase shift measurement diagram wherein three overlapping antenna patterns are presented in polar coordinates.

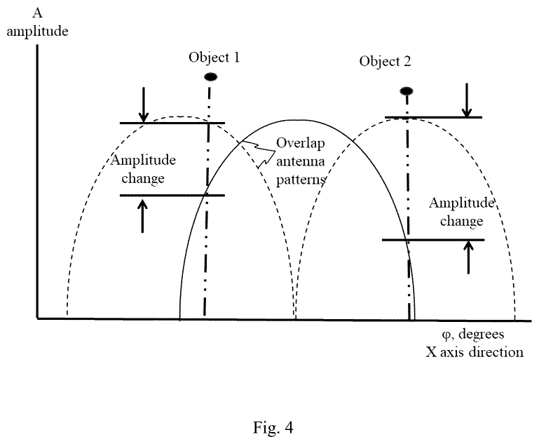

FIG. 5 shows diagram for recording digital hologram of a 3 dimensional object.

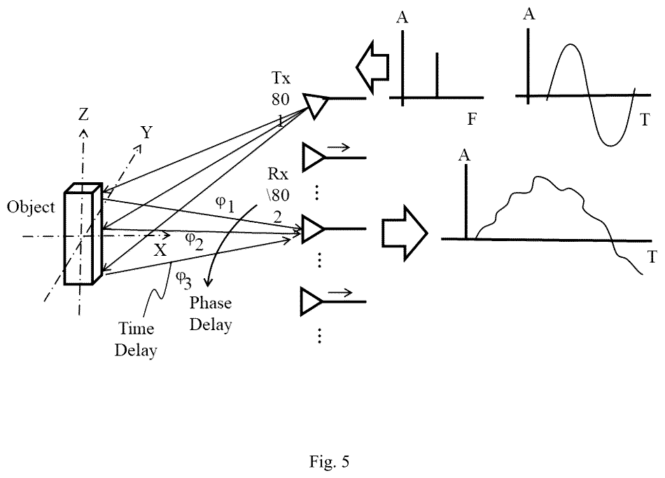

FIG. 6 demonstrates the transformation of digital signals from data matrix to frequency domain and real-time imaging on display.

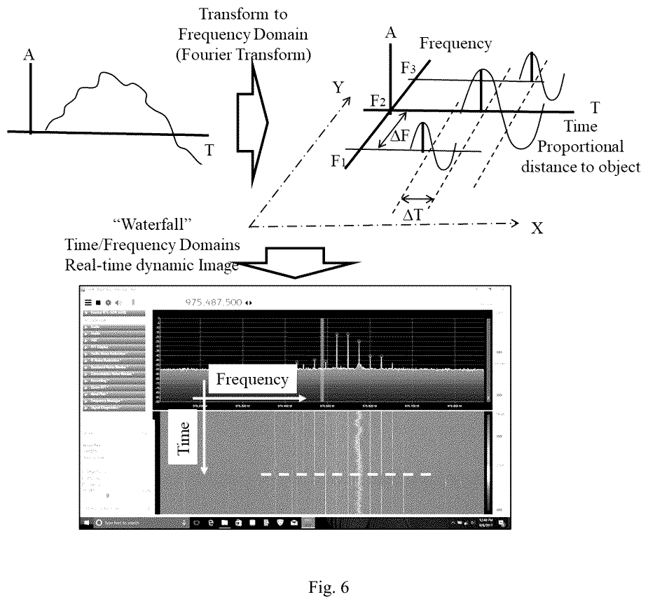

FIG. 7 shows a diagram of Inverse Fourier Transform to time domain and reconstruction of 3-dimensional image of object.

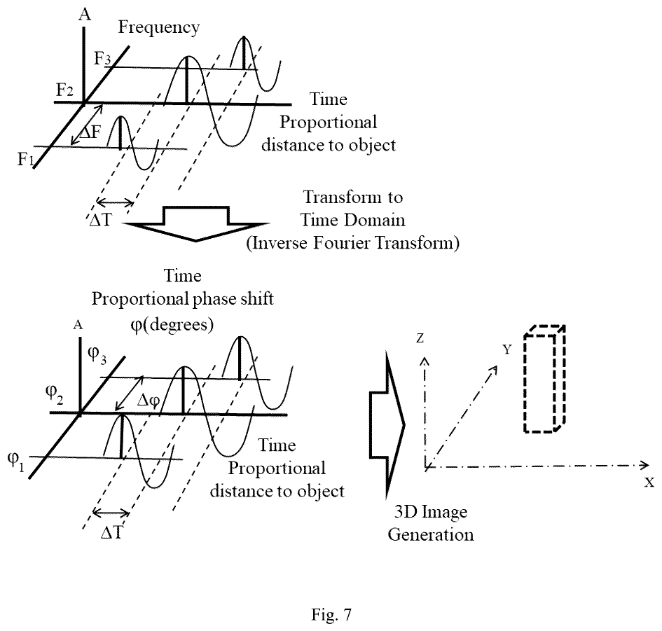

FIG. 8 shows planar antenna array with overlapping antenna patterns for imaging a wide area of observation.

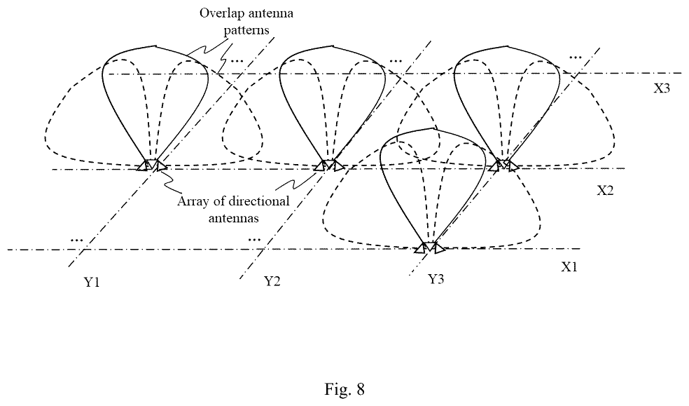

FIG. 9 shows a diagram demonstrating directional antenna arrays with two-axis and three-axis positioning.

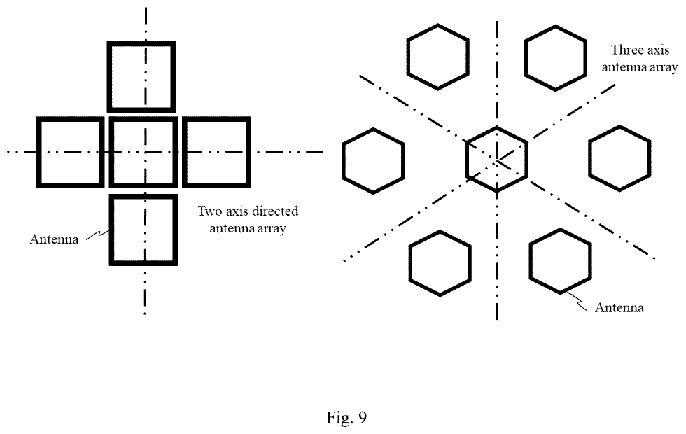

FIG. 10 shows block-diagram of first embodiment of RF enhanced medical imaging and identification system.

FIG. 11 shows of block-diagram of second embodiment of RF enhanced medical imaging and identification system.

FIG. 12 shows block-diagram of embodiment of RF enhanced medical imaging and identification system with separate signal generator.

FIG. 13 shows block-diagram of embodiment of RF enhanced medical imaging and identification system with separate signal generator and separate reference antenna.

FIG. 14 explanation presents imaging method by sequence of operations.

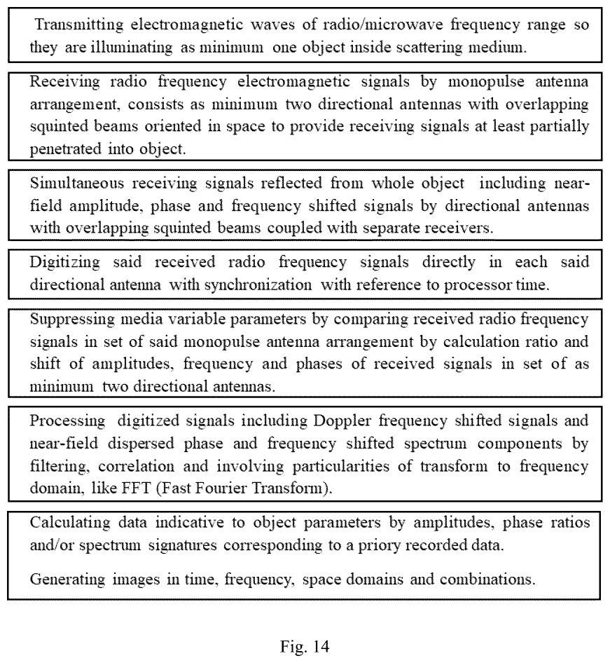

### DETAILED DESCRIPTION OF THE INVENTION

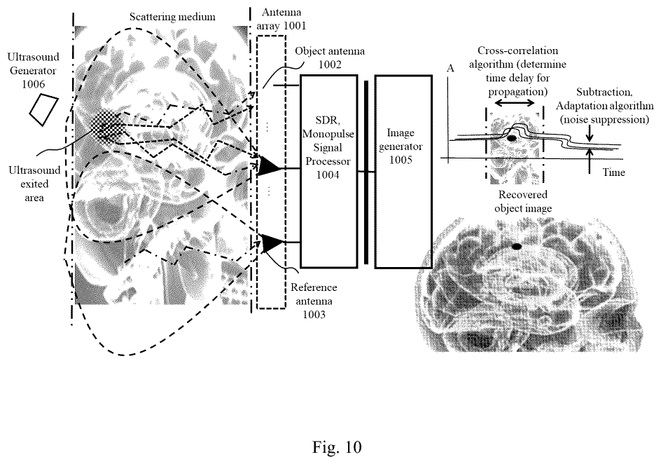

Block-diagram of first embodiment of RF enhanced medical imaging and identification system with ultrasound focusing on the limited area of observation and imaging is presented in FIG. 10. System comprising at least one antenna array 1001 comprising as minimum one object antenna 1002 and as minimum one reference antenna 1003, software defined radio (SDR) for transmitting, receiving and digitizing signals coupled with monopulse signal processor 1004 and image generator 1005. Said antenna array arranged as array of directional antennas covering wide area of observation with staring, not scanning, overlap in as minimum one axis antenna patterns creating antenna sub-arrays in each axis. SDR comprising transmitting means and multiple separate receiving channels with signal conditioning circuits including voltage or current limiters, anti-aliasing circuits, Automatic Gain Control (AGC) means, directional coupler with signal detector and analog-to-digital converter.

Each directional antenna covering whole area or sub-sector of area of observation and coupled with separate receiving cannel providing fast continuous parallel processing of signals for receiving maximum information from all covering area simultaneously. Monopulse signal processor 1004 arranged as multi-channel processor connected with image generator 1005 and comprising memory for real time recording digital hologram, synchronization means and at least one monopulse processor connected to each antenna sub-array by directional coupler with signal detector for creating monopulse subarrays.

Monopulse subarrays comprising means for one or multi-axis processing of all signals in receiving channels as ratio of amplitudes and/or phase shift of signals for direction finding and one-iteration adapting for clutter suppressing or decrease transferring scattering medium influence to receiving chain parameters by phase shift in subarray of neighboring directional antennas with overlap antenna patterns, wherein application of signals from reference antennas providing highest directional accuracy and better clutter/noise and media influence suppression.

Said multi-channel processor 1004 comprised means for transform, correct and filter received signals from time domain to frequency domain and space one axis and/or multi-axis domain to increase image resolution and number of parameters for object recognition. Cross-correlation algorithm allows to adjust and determine time delay for propagation. Subtraction and adaptation algorithm provides noise suppression. Focused ultrasound exited area 1006 allows to limit area of observation for more accurate imaging object position. Said antenna array 1001, receiving and processing means arranged for receiving additional Doppler shifted by ultrasound and diffracted spectrum components in near field consists of information about objects in limited by ultrasound excited area, objects content by spectrum signature and object shape. Said image generator 1005 arranged for generating one or multi-axis images in time, frequency, space, or combined domains. SDR with monopulse processor 1004, image generator 1005 and ultrasound signal generator 1006 are connected by digital interface arranged as universal serial bus (USB) or microwave and/or fiber optic waveguides to signal processor or wireless (not shown).

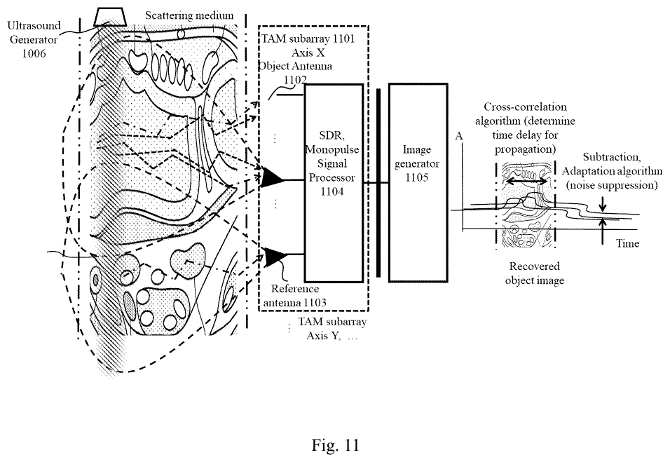

FIG. 11 shows block-diagram of second embodiment of RF enhanced medical imaging and identification system with ultrasound illuminating of slice of imaging. System comprising at least one transceiver antenna module 1101 comprising as minimum one object antenna 1102 and as minimum one reference antenna 1103, software defined radio (SDR) for transmitting, receiving and digitizing signals coupled with monopulse signal processor 1104 and signal generator 1105. Said antenna array arranged as array of directional antennas covering wide area of observation with staring, not scanning, overlap in as minimum one axis antenna patterns creating antenna sub-arrays in each axis. SDR 1104 comprising transmitting means and multiple separate receiving channels with signal conditioning circuits including voltage or current limiters, anti-aliasing circuits, AGC means, directional coupler with signal detector and analog-to-digital converter. Each directional antenna covering whole area or sub-sector of area of observation and coupled with separate receiving cannel providing fast continuous parallel processing of signals for receiving maximum information from all covering area simultaneously. Monopulse signal processor 1104 arranged as multi-channel processor connected with image generator 1105 and comprising memory for real time recording digital hologram, synchronization means and at least one monopulse processor connected to each antenna sub-array by directional coupler with signal detector for creating monopulse subarrays. Cross-correlation algorithm allows to adjust and determine time delay for propagation. Subtraction and adaptation algorithm provides noise suppression. Ultrasound generator 1106 exiting some limited area and allows to limit area of observation for more accurate imaging object/objects position. Said antenna array 1101, receiving and processing means arranged for receiving additional Doppler shifted by ultrasound and diffracted spectrum components in near field consists of information about objects in limited by ultrasound excited area, objects content by spectrum signature and object shape. Said image generator 1105 arranged for generating one or multi-axis images in time, frequency, space, or combined domains. SDR with monopulse processor 1104, image generator 1105 and ultrasound signal generator 1006 are connected by digital interface arranged as universal serial bus (USB) or microwave and/or fiber optic waveguides to signal processor or wireless (not shown).

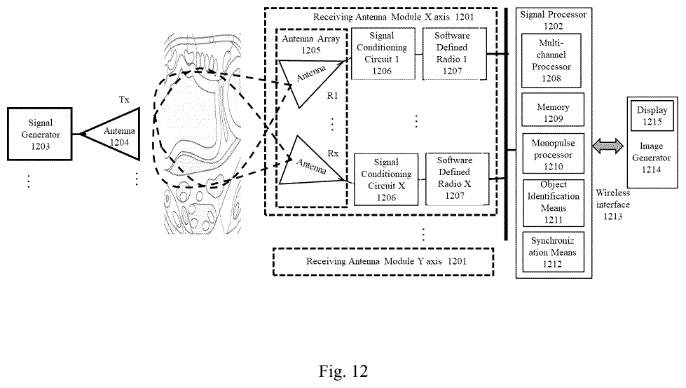

FIG. 12 shows block-diagram of embodiment of RF enhanced medical imaging and identification system with separate signal generator. System comprising receiving antenna module 1201, signal processor 1202 and separate signal generator 1203 with transmitting antenna 1204. Receiving antenna module 1201 comprising antenna array 1205, wherein each antenna coupled with signal conditioning circuit 1206 and SDR 1207. Signal processor 1202 comprising multi-channel processor 1208, memory 1209, monopulse processor 1210, object identification means 1211 and synchronization means 1212. Signal processor 1201 connected by wireless interface 1213 with imaging generator 1214 and display 1215.

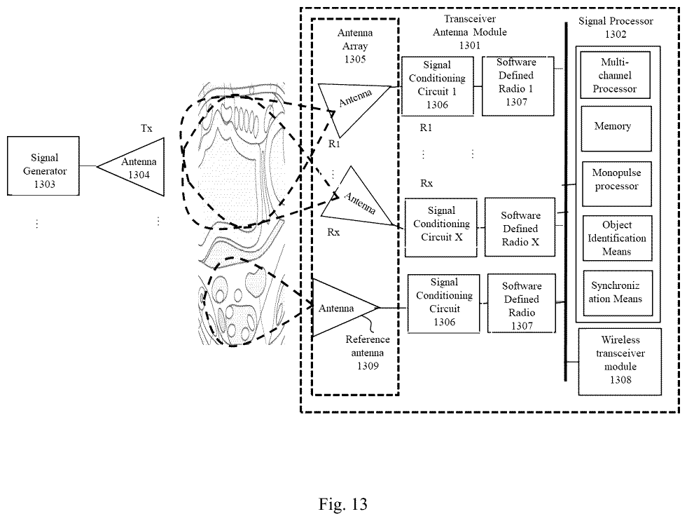

FIG. 13 shows block-diagram of embodiment of RF enhanced medical imaging and identification system with separate signal generator and separate reference antenna. System comprising receiving antenna module 1301, signal processor 1302 and separate signal generator 1303 with transmitting antenna 1304. Receiving antenna module 1301 comprising antenna array 1305, wherein each antenna coupled with signal conditioning circuit 1306 and SDR 1307. Signal processor 1302 comprising multi-channel processor 1308, memory 1309, monopulse processor 1310, object identification means 1311 and synchronization means 1312. Signal processor 1301 connected by wireless interface 1313 with imaging generator 1314 and display 1315. Separate reference antenna 1316 added for suppressing signals scattered by medium or other noises.

### REFERENCE NUMBERS

1001—Array of directional antennas
1002—Object antenna
1003—Reference antenna
1004—SDR, Monopulse signal processor
1005—Image generator
1006—Ultrasound generator
1101—Antenna array
1102—Object antenna
1103—Reference antenna
1104—SDR, Monopulse signal processor
1105—Image generator
1106—Ultrasound generator
1201—Receiving antenna antenna module
1202—Signal processor
1203—Signal generator
1204—Transmitting antenna
1205—Antenna array
1206—Signal conditioning circuit
1207—SDR
1208—Multi-channel processor
1209—Memory
1210—Monopulse processor
1211—Object identification means
1212—Synchronization means
1213—Wireless interface
1214—Image generator
1215—Display
1301—Transceiver Antenna Module
1302—Signal processor
1303—Signal generator
1304—Transmitting antenna
1305—Antenna array
1306—Signal conditioning circuit
1307—SDR
1308—Wireless interface
1309—Reference antenna

### OPERATION

Method of RF enhanced medical imaging and identification system comprising transmitting and receiving of electromagnetic signals means provided by one directional antenna or directional antenna array. Non-scanning transmitting means illuminating entire area of observation or subdivided sectors. Reflected signals simultaneously receiving from whole object or multiple objects within all area or observation or each subdivided sector by set of directional antennas with overlap antenna patterns distributed in one axis, quadrature or multi-axis directions. Processing of received by directional antennas signals providing by multiple separate receiver chains coupled to each receiving antenna. Transmitting power and gain of receiver chains controlling separately in each subdivided sectors by automatic gain control circuit. Automatic gain control circuits allow to simultaneous detection of small range targets with high amplitude reflected targets and targets with small, reflected signals. All transmitting and receiving signals are synchronized by microwave or/and optical means directly in transceiver antenna modules.

Monopulse systems can be continuous waves or pulsed [3]. In continuous wave radars with continuous object or multiple objects observation and integration of the received signals lead to increased object information and image resolution as result. Simultaneous correlation and integration of thousands of signals per second from each point of surveillance allows not only for the detection of low-level signals but can help recognize and classify objects by using diversity signals, polarization modulation, and intelligent processing. Non-scanning monopulse system allows dramatic decrease in transmitting power and at the same received information increasing by integrating 2-3 orders more signals than regular scanning systems.

Synchronizing of signals directly in antennas provide high accuracy amplitude and phase measurement. Non scanning antenna array is phase/frequency independent and can be multi-frequency, multi-function.

Multi-axis processed signals from receiving antennas can be applied for detection and identification of objects in each separate set of receiving antennas and for generating alarm signal and multi-axis signals proportional object position, shape, size, impedance, identification data.

Monopulse means can consist filters in identification circuits for separation and suppress scattering signals, object signals form background noise, scattering medium, identification of objects and medium components.

### CONCLUSION

Cover of whole imaging object and continuous illumination of object providing by staring array of directional antennas increasing imaging resolution and probability of object identification.

Multi-axis distribution of overlap antennas allows to receive maximum information about shape, size and content of imaging object.

Coupling of each directional antenna with separate receiver channel allows receive information about whole object simultaneously and much faster.

Monopulse processing of signals and receiving signals from reference sub-set of antennas with overlap antenna patterns provides highest directing accuracy and resolution and better clutter/noise and scattering media influence suppression. Separate controlling of transmitting power and gain of receiver chains in each subdivided sectors by automatic gain control circuit provides extension of dynamic range of system. Automatic gain control circuits also allow to simultaneous detection of objects with small reflecting signals.

Digitizing and synchronization of all receiving signals by microwave or/and optical means directly on directional antennas allows loose distribution of antennas without complicate phase adjustment matrixes.

1. Method for enhanced medical imaging and identification of objects by receiving of electromagnetic waves transmitted and reflected from as minimum one object and scattering medium comprising:
transmitting electromagnetic waves of radio/microwave frequency range so they are illuminating as minimum one object and scattering medium;
receiving radio/microwave frequency electromagnetic signals by monopulse antenna arrangement, consists as minimum two directional antennas with overlapping squinted beams oriented in space to provide receiving signals reflected from as minimum one object and at least partially penetrating it;
simultaneous conditioning receiving signals including near-field amplitude, phase and frequency components shift by directional antennas with overlapping squinted beams, wherein each directional antenna coupled with separate receiver;
direct digitizing said received radio/microwave frequency signals in each said directional antenna with synchronization with reference to processor time and recording real time data matrix or digital hologram representing reflected from possible objects and scattering medium wavefront;
virtual scanning wavefront signals by processing of all digitized signals including Doppler frequency shifted signals and near-field dispersed phase and frequency shifted spectrum components by filtering, correlation and involving particularities of time-frequency domains transform like FFT (Fast Fourier Transform);
determine objects and medium components position and shape, and suppressing scattering medium variable parameters by comparing received radio/microwave frequency signals in set of said monopulse antenna arrangement by calculation ratio and shift of amplitudes, frequency and phases of received signals in set of as minimum two directional antennas;
identification and color coding of objects and scattering medium components by filtering spectrum signatures comprising received near field dispersed frequency components;
generating objects image by invert transformation of real time 2- or 3-dimensional digital hologram data.

2. Method for enhanced medical imaging and identification of claim 1 wherein objects position and shape determined by scanning ultrasound signals beam inside area covered by transmitted and received electromagnetic waves and filtering of Doppler shifted signals.

3. RF enhanced imaging and identification radar comprising at least one antenna array, means for transmitting electromagnetic signals, means for receiving radar signals and means for processing the radar signals wherein:
said antenna array arranged as array of directional antennas covering area of observation with overlap in as minimum one axis antenna patterns creating antenna sub-arrays in each axis;
means for transmitting radar signals comprising as minimum one phase-locked loop, signal generator and controllable power amplifier connected with transmitting antenna covering whole area or sub-sector of area of observation;
means for receiving radar signals comprising multiple separate receiving channels with signal conditioning circuit including voltage or current limiters, anti-aliasing circuits, Automatic Gain Control (AGC) means, directional coupler with signal detector and analog-to-digital converter;
each directional antenna covering whole area or sub-sector of area of observation and coupled with separate receiving cannel providing fast continuous parallel processing of signals for receiving maximum information from all covering area simultaneously;
means for processing the radar signals comprising multi-channel processor, image generator, memory for real time recording digital hologram, synchronization means and at least one monopulse processor connected to each antenna sub-array by directional coupler with signal detector for creating monopulse subarrays;
monopulse subarrays comprising means for one or multi-axis processing of all signals in receiving channels as ratio of amplitudes and/or phase shift of signals for direction finding and one-iteration adapting for clutter suppressing or decrease transferring media influence to receiving chain parameters by phase shift in subarray of neighboring directional antennas with overlap antenna patterns, wherein application of signals from reference antennas providing highest directional accuracy and better clutter/noise and media influence suppression;
said multi-channel processor comprised means for transform, correct and filter received signals from time domain to frequency domain and space one axis and/or multi-axis domain to increase image resolution and number of parameters for object recognition;
said antenna array, receiving and processing means arranged for receiving additional Doppler shifted and diffracted spectrum components consists of information about moving objects, objects content by spectrum signature and object shape;
said image generator arranged for generating one or multi-axis images in time, frequency, space or combined domains;
means for transmitting radar signals, means for receiving radar signals and means for processing the radar signals are connected by digital interface arranged as universal serial bus (USB), microwave and/or fiber optic waveguides or wireless to signal processor.

4. RF enhanced imaging and identification radar of claim 3 comprising at least one antenna array, means for transmitting electromagnetic signals, means for receiving radar signals and means for processing the radar signals wherein:
said antenna array arranged as array of directional antennas covering area of observation with overlap in as minimum one axis antenna patterns creating antenna sub-arrays in each axis;
means for transmitting radar signals and means for receiving radar signals comprising Software Defined Radios (SDR) with multiple separate transmitting/receiving channels and with analog-to-digital digital-to analog converters;
each directional antenna covering whole area or sub-sector of area of observation and coupled with SDR separate receiving cannel providing fast continuous parallel processing of signals for receiving maximum information from all covering area simultaneously;
means for processing the radar signals comprising multi-channel processor, image generator, memory for real time recording digital hologram, synchronization means and at least one monopulse processor connected to each antenna sub-array by directional coupler with signal detector for creating monopulse subarrays;
monopulse subarrays comprising means for one or multi-axis processing of all signals in receiving channels as ratio of amplitudes and/or phase shift of signals for direction finding and one-iteration adapting for clutter suppressing or decrease transferring media influence to receiving chain parameters by phase shift in subarray of neighboring directional antennas with overlap antenna patterns, wherein application of signals from reference antennas providing highest directional accuracy and better clutter/noise and media influence suppression;
said multi-channel processor comprised means for transform, correct and filter received signals from time domain to frequency domain and space one axis and/or multi-axis domain to increase image resolution and number of parameters for object recognition;
said antenna array, receiving and processing means arranged for receiving additional Doppler shifted and diffracted spectrum components consists of information about moving objects, objects content by spectrum signature and object shape;
said image generator arranged for generating one or multi-axis images in time, frequency, space or combined domains;
means for transmitting radar signals, means for receiving radar signals and means for processing the radar signals are connected by digital interface arranged as universal serial bus (USB), microwave and/or fiber optic waveguides or wireless to signal processor.

5. RF enhanced imaging and identification radar of claim 3, wherein said antenna array coupled with transmitting and receiving means are arranged as concave, convex, cylindric full/hemi sphere modules consisting of plurality of antenna elements which forming directional antennas coupled with synchronized transmitting and receiving means which may be distributed around observation area and connected wireless.

6. RF enhanced imaging and identification radar of claim 3, wherein said means for transmitting, receiving and processing means are arranged for simultaneous transmitting, receiving, and processing modulated signals or signals on a few different frequencies (multi-frequency signals) and different modes of signals and comprising corresponding arranged directional antennas, anti-aliasing circuits and filtering means in each transmitter and receiving chain.
